(19) United States
(12) Patent Application Publication
(10) Pub. No.: US 2018/0078420 A1
(43) Pub. Date: Mar. 22, 2018

(54) PROTECTIVE EYEWEAR WITH DUAL-MODE SIDEARMS

(71) Applicant: 3M INNOVATIVE PROPERTIES COMPANY, St. Paul, MN (US)

(72) Inventors: Hans O.E. Gustafsson, Värnamo (SE); Anton J. Hjalmarsson, Bredaryd (SE)

(21) Appl. No.: 15/558,740

(22) PCT Filed: Mar. 31, 2016
(86) PCT No.: PCT/US2016/025296

§ 371 Date: Sep. 15, 2017

Related U.S. Application Data
(60) Provisional application No. 62/144,567, filed on Apr. 8, 2015.

(51) Int. Cl. A61F 9/02; G02C 5/14; G02C 5/20; G02C 5/22; G02C 11/00

(52) U.S. Cl. CPC A61F 9/029; G02C 5/143; G02C 11/00; G02C 5/2263; G02C 5/20

(57) ABSTRACT

A protective eyewear with dual-mode sidearms allows the eyewear to be worn in the presence or absence of earmuff-type hearing-protection apparatus. The protective eyewear comprises: an eyeshield; a first sidearm that is pivotally connected to the eyeshield by a first primary pivotal connection that is located proximate a first major end of the eyeshield; and a second sidearm that is pivotally connected to the eyeshield by a second primary pivotal connection that is located proximate a second, opposing major end of the eyeshield, wherein at least a rearward portion of a second, rear part of each sidearm comprises a laterally-outward-and-downward-facing major surface that is a concave surface, wherein the primary pivotal connection of each sidearm is configured so that each sidearm can be pivotally moved between at least a first, lowered configuration and a second, raised configuration, and wherein the sidearms are configured so that when the protective eyewear is worn in combination with an earmuff-type hearing-protection apparatus and when both sidearms are in the second, raised configuration, the rearward portion of the rear part of each sidearm rests atop a junction between an uppermost portion of a compliant seal of an earmuff of the hearing-protection apparatus, and a side of a user's head.

### BACKGROUND

Protective eyewear is often worn in situations in which protection from e.g. particulate debris, splashing liquids, or the like, is desired.

### SUMMARY

In broad summary, herein is disclosed protective eyewear with dual-mode sidearms. These and other aspects of the invention will be apparent from the detailed description below. In no event, however, should this broad summary be construed to limit the claimable subject matter, whether such subject matter is presented in claims in the application as initially filed or in claims that are amended or otherwise presented in prosecution.

### BRIEF DESCRIPTION OF THE DRAWINGS

FIG. 1 is a rear-side perspective view of an exemplary protective eyewear.

FIG. 2 is a rear-side perspective view of the exemplary protective eyewear of FIG. 1, further illustrating an exemplary hearing-protection earmuff of an earmuff-type hearing-protection apparatus.

FIG. 3 is a perspective view, from the right side and from slightly above, of the exemplary protective eyewear and exemplary earmuff of FIG. 2.

FIG. 4 is a front perspective view in partial cross-section, view of an exemplary sidearm of an exemplary protective eyewear.

FIG. 5 is an idealized cross-sectional front view of an exemplary sidearm of a protective eyewear, and an exemplary hearing-protection earmuff of a hearing-protection apparatus, when in position against the side of a user's head.

FIG. 6 is a side view of an exemplary protective eyewear, with a sidearm of the eyewear in a lowered configuration.

FIG. 7 is a side view of an exemplary protective eyewear, with a sidearm of the eyewear in a raised configuration.

Like reference numbers in the various figures indicate like elements. Some elements may be present in identical or equivalent multiples; in such cases only one or more representative elements may be designated by a reference number but it will be understood that such reference numbers apply to all such identical elements. Unless otherwise indicated, all figures and drawings in this document are not to scale and are chosen for the purpose of illustrating different embodiments of the invention. In particular the dimensions of the various components are depicted in illustrative terms only, and no relationship between the dimensions of the various components should be inferred from the drawings, unless so indicated.

As used herein as a modifier to a property or attribute, the term “generally”, unless otherwise specifically defined, means that the property or attribute would be readily recognizable by a person of ordinary skill but without requiring a high degree of approximation (e.g., within +/−20% for quantifiable properties). The term “substantially”, unless otherwise specifically defined, means to a high degree of approximation (e.g., within +/−10% for quantifiable properties). The term “essentially” means to a very high degree of approximation (e.g., within plus or minus 2% for quantifiable properties; it will be understood that the phrase “at least essentially” subsumes the specific case of an “exact” match. However, even an “exact” match, or any other characterization using terms such as e.g. same, equal, identical, uniform, constant, and the like, will be understood to be within the usual tolerances or measuring error applicable to the particular circumstance rather than requiring absolute precision or a perfect match. All references herein to numerical parameters (dimensions, ratios, and so on) are understood to be calculable (unless otherwise noted) by the use of average values derived from a number of measurements of the parameter, particularly for the case of a parameter that is variable.

### DETAILED DESCRIPTION

### Glossary

For clarity of description, the following terminology is used:

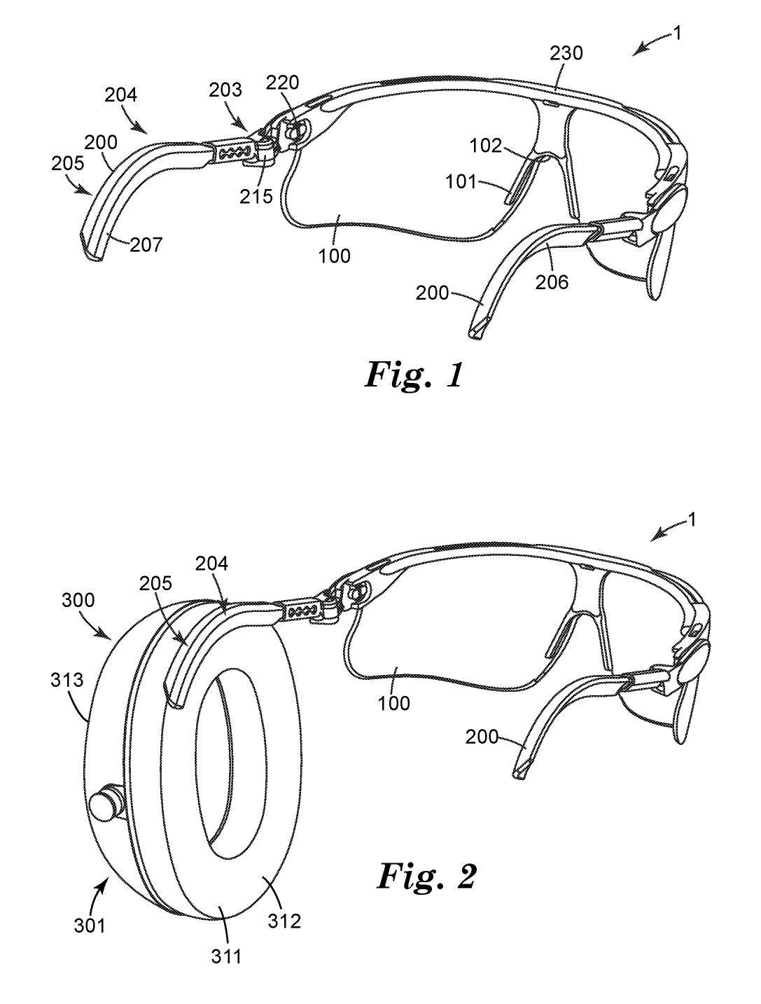

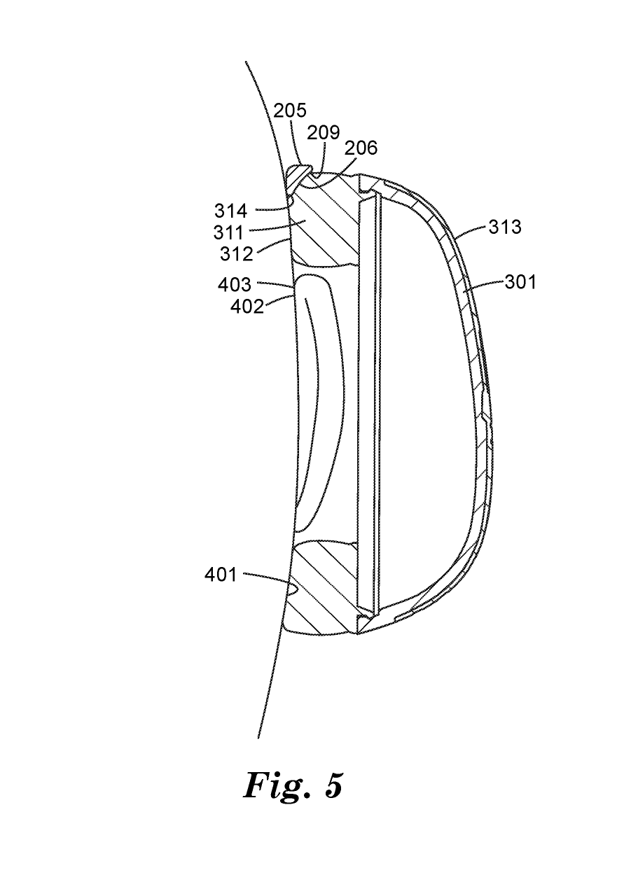

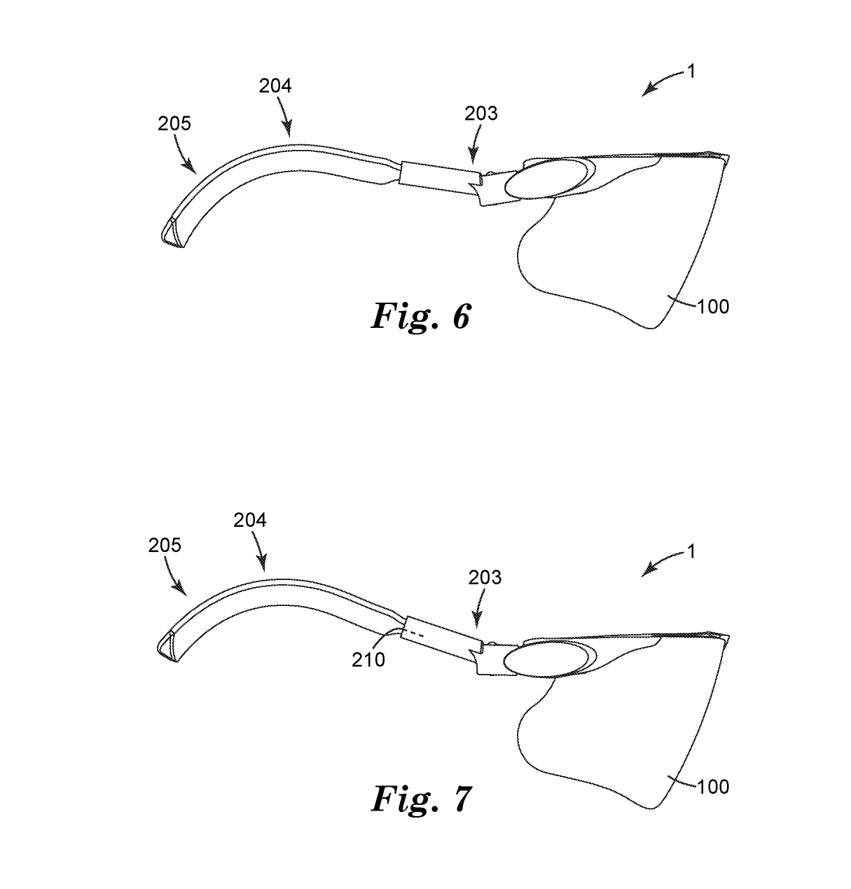

Terms such as front, frontward, forward, etc. refer to a direction that is toward a scene to be viewed (e.g. toward a light source, a workpiece, and so on) when protective eyewear 1 is worn by a user. Terms such as rear, rearward, etc., refer to a direction that is generally away from the front direction; i.e., toward the back of the user's head. Terms such as upward, above, top, etc., are used in the conventional sense as meaning toward the top of the user's head. Terms such as downward, below, under, etc. are used in the conventional sense as meaning away from the top of the user's head and toward the user's feet. (All geometric and anatomic descriptions herein are understood to presume that the user is in an upright (e.g. standing) position with the head held level.) Terms such as laterally inward mean at least generally toward the sagittal plane of the user's head; laterally outward means away from the sagittal. By way of specific examples, FIG. 1 is view from the rear (and slightly from the right side) of the protective eyewear; FIG. 5 is a (cross-sectional) view from the front of the protective eyewear, and FIG. 6 is a view from the side. Up/down (u/d) and inward/outward (i/o) axes are included in some Figures for added clarity.

Protective eyewear 1 is shown in exemplary embodiment in rear-side view in FIG. 1 and in side view in FIG. 6. Protective eyewear 1 comprises eyeshield 100 (described in detail later herein) and first and second sidearms 200 (first and second sidearms will often be mirror images of each other). First sidearm 200 is pivotally connected to eyeshield 100 by a first primary pivotal connection 220 that is proximate a first major end of eyeshield 100; second sidearm 200 is pivotally connected to eyeshield 100 by a second primary pivotal connection 220 that is proximate a second major opposing end of eyeshield 100. (As will be made clear later herein, this characterization does not require that a sidearm must necessarily be connected exactly to a terminal end/edge of the eyeshield, nor does it require that the sidearm must necessarily be e.g. connected directly to the transparent portion of the eyeshield.)

Sidearms 200 each have a first, front part 203 and a second, rear part 204, which are slidably engaged with each other as described later herein. (For convenience of description, features of sidearms may be described with reference to a single sidearm; it will be understood that such descriptions will typically apply to both sidearms.)

Rear part 204 of each sidearm includes a rearward portion 205 (e.g., an “earhook”) that exhibits a generally arcuate shape when viewed from the side (e.g. as in FIGS. 6 and 7). Each rearward portion 205 is shaped and configured so that it can be “hooked” over the top of a user's ear when protective eyewear 1 is worn in a conventional manner. However, as presented later herein in detail, protective eyewear 1 is designed and configured so that in addition to being wearable in a conventional manner (specifically, in the absence of an earmuff-type hearing-protection apparatus), protective eyewear can, if desired, be worn in combination with a earmuff-type hearing-protection apparatus 300. An earmuff-type hearing-protection apparatus 300 will comprise first and second hearing-protection earmuffs 301, that are connected with each other by a headband. For convenience of presentation, one of the earmuffs 301, and the headband, are omitted from e.g. FIG. 2; however, the ordinary artisan will understand that the second earmuff would likely appear as a mirror image of the depicted earmuff. Features of hearing-protection apparatus 300 are described in detail later herein.

Protective eyewear 1, in particular first and second sidearms 200 thereof, is thus designed and configured to provide dual-mode functionality. By this is meant that in a first mode of use eyewear 1 can be worn in the absence of an earmuff-type hearing-protection apparatus. In such a case the rearward (earhook) portion 205 of each sidearm rests atop the respective ear of the user. Specifically, it rests atop the uppermost portion of the junction 402 of the user's pinna 400 with the side of the user's head 401 (which location is pointed out in FIG. 5) as with any conventional eyewear. In a second mode of use eyewear 1 can be worn in the presence of an earmuff-type hearing-protection apparatus (e.g. of the general type exemplified by apparatus 300). The ordinary artisan will appreciate that an earmuff-type hearing-protection apparatus will comprise first and second hearing-protection earmuffs 301 that each comprise a hard shell 313 with a compliant seal 311 (e.g., a foam cushion) extending around the perimeter of the hard shell and protruding inwardly therefrom so as to contact the side of the user's head when the hearing-protection apparatus is worn. (The ordinary artisan will know that the purpose of such a compliant seal is to minimize any leakage of sound between the seal and the side of the user's head, without exerting excessive pressure on the user's head.) In the second mode of use of protective eyewear 1, the rearward portion 205 of each sidearm will rest atop an upward portion of compliant seal 311 of the hearing-protection earmuff. Specifically, it will rest atop location 314 which is the uppermost location at which the inward surface 312 of compliant seal 311 abuts against the side of the user's head 401), as shown in exemplary embodiment in FIG. 5. Although not visible in the cross-sectional view of FIG. 5, it will be appreciated that location 314 upon which rearward portion 205 of the sidearm rests, will typically take the form of an arch along the upward portion of compliant seal 311, rather than being e.g. a single point of contact.

It will be appreciated that this second mode of use allows protective eyewear 1 to be worn in combination with an earmuff-type hearing-protection apparatus, without the sidearms of the protective eyewear disrupting the acoustic seal that is desirably maintained between all portions of the compliant seal of the earmuff, and the side of the user's head.

In aid of this dual-mode functioning, the rearward portion 205 of each sidearm exhibits a first major surface 206 that is concave in shape and that faces generally laterally outward and downward. (This can be seen most easily in FIG. 6, and in particular in FIG. 4, which is a cross-sectional isolated view of a section of a rearward portion 205 of a sidearm, viewed from the front.) Alternatively phrased, surface 206 exhibits a laterally-outward-extending overhang. It will be appreciated that the curvature and orientation of major surface 206 allows rearward portion 205 of sidearm 200 to fit snugly against an uppermost portion of inward-facing surface 312 of compliant seal 311; specifically, it allows it to fit into a space between the compliant seal and the user's head, which space is just above the uppermost junction 314 of compliant seal 311 with the side of the user's head 401 as shown in FIG. 5. The ordinary artisan will appreciate that inward pressure exerted on compliant seal 311 (e.g. as motivated by an inward-biasing force imparted by the headband) will cause the inward-facing surface 312 of the compliant seal 311 to conform against the side of the user's head (e.g., to flatten slightly against the head) and also will cause the compliant seal to deform (bulge) slightly upward so that the outer surface of the seal (e.g. the outer surface of the uppermost portion of the seal) assumes a slightly convex shape (as shown in FIG. 5). The concave curvature and orientation of surface 206 of the rearward portion of each sidearm may thus be chosen so as to nestle in a crevice between this bulged portion of the compliant seal and the side of the user's head. This allows rearward portion 205 of each sidearm to fit securely atop junction 314 between the compliant seal 311 and the side of the user's head 401. It will be appreciated that the compliance of seal 311 will provide that portions of the seal below rearward portion 205 of each sidearm will still be able to contact the side of the user's head so as to minimize the chance of any leakage of sound, as shown in idealized manner in FIG. 5. This can be contrasted with conventional methods in which conventional protective eyewear is worn with an earmuff-type hearing-protection apparatus, in which case the sidearm often passes between the inward face of the compliant seal and the user's head (and penetrates into the interior space defined by the earmuff and the side of the user's head) in such a manner that the seal cannot conform tightly enough around the sidearm to minimize leakage of sound.

The concave shape of major surface 206 may be characterized by a radius of curvature. In various embodiments, laterally-outward-and-downward-facing concave surface 206 of the rearward portion 205 of the rear part 204 of each sidearm, may exhibit an average radius of curvature (measured along the extent of the rearward portion 205 along which an identifiable concave surface 206 is present) of at least about 6, 8, 10, 12, 14 or 16 mm. In further embodiments, such an average radius of curvature may be at most about 24, 22, 20, 18, 16, or 14 mm.

In some embodiments (e.g. in the exemplary embodiments of FIGS. 1, 4 and 6, the radius of curvature may be relatively constant along the length of surface 206. Thus in some embodiments, laterally-outward-and-downward-facing concave surface 206 of the rearward portion 205 of the rear part 204 of each sidearm, exhibits a radius of curvature that does not deviate more than plus or minus 20%, 10%, or 5%, from an average value over the extent (length) of rearward portion 205.

In some embodiments, at least the rearward portion 205 of the second, rear part 204 of each sidearm may comprise a laterally-inward facing second major surface 207 that is a generally flat surface exhibiting a major plane that is at least substantially vertically oriented when the eyewear is worn by a user. Arrangements of this type, shown in exemplary embodiment in FIGS. 4 and 5, can advantageously provide that this surface of rearward portion 205 of each sidearm is able to nestle closely against the side of the user's head.

The overall arcuate shape of rearward portion 205 of each sidearm may be chosen to acceptably fit the shape of the pinna of most adult human users, as well as to acceptably fit the shape of the uppermost portion of the compliant seal of many available earmuff-type hearing-protection apparatus. Such an arrangement can be characterized by the radius of curvature of rearward portion 205, when eyewear 1 is viewed from the side (e.g. as in FIGS. 6 and 7). In various embodiments, when so viewed, rearward portion 205 of the rear part 204 of each sidearm may exhibit an arched shape that exhibits a radius of curvature of from about 25 mm, or 30 mm, to about 40 or 45 mm. If desired, the rearmost few millimeters of rearward portion 205 of each sidearm may bend slightly inwards (e.g., toward the sagittal plane of a user's head); however, in many embodiments (e.g. as shown in FIG. 1) no such inward bending may be present.

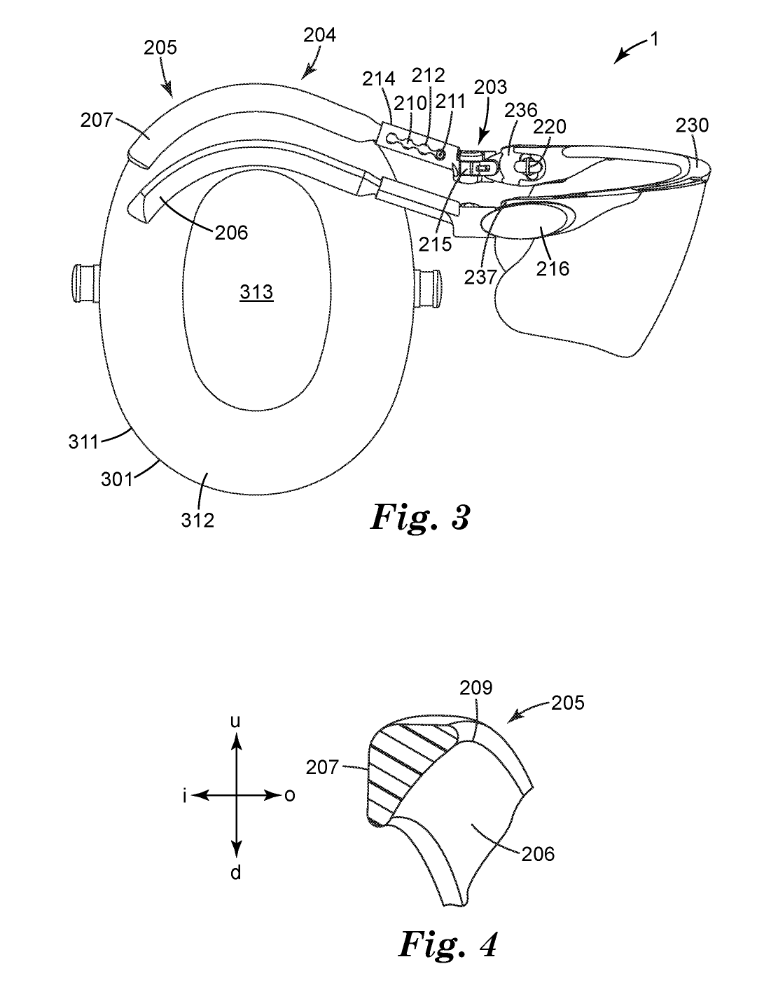

In further aid of dual-mode functioning as described herein, at least the rear part 204 of each sidearm 200 is pivotally connected to the eyeshield 100 so that at least the rear part of each sidearm can pivot upward and downward relative to the eyeshield. In many embodiments, it may be convenient that generally, substantially, or essentially the entire length of sidearm 200 (e.g. including the front part 203 in addition to the rear part 204) may be pivotally connected to eyeshield 100 so that, e.g., essentially the entire length of the sidearm can be pivoted upward and downward. For example, in arrangements of the general type illustrated in FIGS. 3, 6 and 7, sidearm 200 is pivotally connected to an eyeshield 100 by primary pivotal connection 220 so that essentially the entire length of the sidearm 200 can be pivoted upward and downward along an arc whose center of rotation is located at pivotal connection 220.

Such arrangements provide that at least the rearward portion 205 of each sidearm can be raised upward to a position (as shown e.g. in FIG. 7) relative to eyeshield 100 (and relative to the ears of a user, when protective eyewear 1 is worn) that is exaggeratedly upward when compared to the sidearms of most conventional protective eyewear. (In other words, most conventional protective eyewear positions a sidearm at an angle typical of that shown in FIG. 6, with any pivotability serving only to provide sufficient range of pivotal motion to compensate for the natural variation in the head shape and geometry of different users.) In the present instance, the rearward portion 205 of each sidearm may be positioned far upwards from such a conventional position, in order to allow the rearward portion 205 of the sidearm to rest atop the junction 314 between the compliant seal 311 of a hearing-protection earmuff and the side of a user's head 401 (as shown in idealized representation in FIG. 5). In other words, in the present design, the “earhook” 205 can rest atop the compliant seal of the hearing-protection earmuff rather than atop the pinna of the user.

The ordinary artisan will appreciate that such a junction 314 between the compliant seal 311 and the side of the user's head 401, may be e.g. up to a centimeter or more above the junction 402 between the pinna and the side of the user's head 401. To provide this range of motion, in various embodiments the primary pivotal connection 220 of a sidearm may allow the sidearm to pivotally move along an upward-downward arc, over a rotation angle of, for example, at least about 5, 10, 15, 20, or 25 degrees. (The rotation may be measured using any suitable reference point e.g. along the rearward portion of the sidearm.)

The primary pivotal connection 220 of each sidearm is configured so that the sidearm can be pivotally moved between at least a first, lowered configuration in which the rearward portion of the rear part of the sidearm is in a maximally downward position (e.g. as depicted in FIG. 6), and a second, raised configuration in which rearward portion of the rear part of the sidearm is in a maximally upward position (e.g., as depicted in FIG. 7). Such arrangements allow that when protective eyewear 1 is worn in combination with an earmuff-type hearing-protection apparatus and when both sidearms are in the second, raised configuration, for each sidearm the rearward portion 205 of the rear part 204 of the sidearm rests atop a junction 314 between an upper portion of a the compliant seal of the earmuff of the hearing-protection apparatus, and a side of the user's head, as discussed earlier and as shown in FIG. 5. (Also as noted before, the upper portion of the compliant seal atop which the rearward portion 205 of the sidearm rests, will typically be an arcuate section of the seal rather than an isolated uppermost point.)

Primary pivotal connection 220 of each sidearm can be of any suitable design that allows the desired amount of pivotal movement (i.e., rotation about the pivotal connection). In some embodiments, each primary pivotal connection 220 may be configured (e.g., may comprise interference features) so that the pivotal movement of the sidearm occurs in discrete increments. Such arrangements may also serve to maintain the sidearm at a particular rotation angle (e.g., in a first, lowered configuration or a second, raised configuration) unless sufficient rotational force is applied by the user to pivotally move the sidearm to a different rotation angle. In some embodiments, a primary pivotal connection may allow its sidearm to be pivotally moved between three configurations: a first, lowered configuration; a second, upward configuration, and a third, intermediate configuration that is intermediate between the first and second configurations. If desired, the sidearm may be moved into more than one intermediate configuration.

The exemplary primary pivotal connection 220 as most easily seen in FIG. 3 relies on a post that protrudes inwardly from front part 203 of sidearm 200, which post passes through an aperture provided in an extending portion 236 of a brow frame 230 (described later). The post and aperture comprise interference features so that the pivotal motion of the sidearm occurs in discrete increments. However, it will be understand that any suitable primary pivotal connection, of any type, may be used.

The arrangements disclosed herein allow the rearward portion 205 of each sidearm 200 to be brought to a vertical position that is higher than conventionally employed. This arrangement may be characterized by the vertical offset between an uppermost point 209 of the laterally-outwardly-and-downwardly-facing major surface 206 of rearward portion 205 (point 209 is most easily seen in FIG. 7), and an uppermost point 403 of the junction 402 between the pinna 400 of a user and the side of the user's head 401. In various embodiments, such a vertical offset may be at least about positive 6, 8, 10, 12, 14, 16, 18, 20, 22, 24, 26, 28, or 30 mm (with a positive offset meaning that point 209 is higher than point 403). Such arrangements may also be characterized by the vertical offset between the above-mentioned uppermost point 209 of the outwardly-and-downwardly-facing major surface 206 of rearward portion 205, and the uppermost point 102 of a nose-rest 101 of eyeshield 100 (an exemplary nose-rest 101 and uppermost point 102 thereof is pointed out in FIG. 1). In various embodiments, such an offset may be at least about (positive) 6, 8, 10, 12, 14, 16, 18, 20, 22, 24, 26, 28, or 30 mm. (All such measurements and characterizations should be made with the protective eyewear fitted to a user in normal fashion, i.e. with the eyewear seated snugly against the nose and with the user's head upright). It is noted that the uppermost point 102 of a nose-rest is merely a convenient reference point for characterizing such a vertical offset; it will be appreciated that when the eyewear is worn, the uppermost point 102 of a nose-rest may not necessarily be in direct contact with the user's nose.

In further aid of dual-mode functioning as disclosed herein, each sidearm 200 may optionally be a telescoping sidearm such that the rear part 204 of the sidearm is movable forward and rearward relative to the front part 203 of the sidearm. In particular, it may sometimes be helpful to move the rear part 204 rearward so that the total length of the sidearm is increased if the “earhook” portion 205 of the rear part 204 of the sidearm is to be fitted atop a compliant seal of a protective earmuff rather than atop the user's pinna. Thus in some embodiments, for each sidearm the first, front part 203 and the second, rear part 204 of the sidearm are slidably connected with each other and are slidably movable relative to each other so that the rear part of the sidearm can be moved rearward relative to the front part of the sidearm to increase the frontward-rearward length of the sidearm by at least about 5, 10, or 15 mm (from its shortest length).

In some embodiments it may be convenient to achieve this by providing that rear part 204 of sidearm 200 includes a frontmost member portion 210 that is slidably insertable into a receiving sleeve 214 that is provided in a rearward portion of front part 203 of sidearm 200. (Such arrangements are most easily visible in FIG. 3.) It will be appreciated that if a frontmost member portion 210 (of rear part 204) of this type is present, it does not form part of the above-described rearward portion 205 of rear part 204. In some embodiments the slidably engagable portions of the front and rear parts (e.g. receiving sleeve 214 and member portion 210) may be friction-fitted together so that rear part 204 of sidearm 200 can be moved to any desired location (relative to the front part of the sidearm) along the available distance of slidable travel, and will tend to stay at that location unless sufficient forward or rearward force is applied by the user to slidably move the rear part of the sidearm to a different location.

In some embodiments, the slidable connection between the first, front part and the second, rear part of the sidearm may comprise interference features (e.g. in the form of complementary features on the front and rear parts of the sidearm) that are configured so that the slidable motion of the rear part relative to the front part occurs in discrete increments. Each interference feature (or pair thereof) can maintain the rear part of the sidearm at a particular location unless sufficient forward or rearward force is applied by the user to slidably move the rear part of the sidearm to a different location relative to the front part of the sidearm. (Exemplary complementary interference features in the form of a short inwardly protruding post 211 that is located on member portion 210 of the rear part 204 of the sidearm, and a series of scallops 212 that are located on an inward wall of receiving sleeve 214 of the front part 203 of the sidearm, as visible in FIG. 3.)

If desired, protective eyewear 1 may comprise optional secondary pivotal connections 215 that allow each sidearm 200 to be pivoted inward so that eyewear 1 can be folded for storage. Thus in some embodiments, each 200 sidearm may further comprise (in addition to the above-described primary pivotal connection 220) a secondary pivotal connection 215 (e.g., a hinged connection) that allows at least the rear part of the sidearm to be rotated inwardly so that the sidearms of the protective eyewear can be placed in an inwardly-folded configuration e.g. for storage. An exemplary secondary pivotal connection (specifically, a hinged connection) 215 is visible in FIG. 3.

In some embodiments as most easily seen in FIGS. 3 and 6, for each sidearm, a forward end of front part 203 of the sidearm may be pivotally connected to the eyeshield by a primary pivotal connection 220; and, a rearward end of front part 203 of the sidearm may be slidably connected to the rear part of the sidearm, both as described above. In the illustrated embodiment of FIGS. 3 and 6, a secondary pivotal (hinged) connection 215 may be provided in a location of the front part of the sidearm that is between the forward end and the rearward end, of the front part. Thus in the illustrated embodiment, first, front part 203 of sidearm 200 is provided by two pieces (a first, forward piece 216 that is pivotally connected to the eyeshield, and a second, rearward piece that comprises sleeve 214), the two pieces being joined together by hinged connection 215. It is emphasized that the particular designs illustrated herein are exemplary. It will be understood that many arrangements are possible while remaining within the scope of the disclosures herein; for example, hinged connection 215 could be located forward of primary pivotal connection 220, if desired. Regardless of the specific design, it will be appreciated that such arrangements provide that the rear part of each sidearm exhibits two-axis pivotability, being pivotal along an upward-downward arc by way of being pivoted about the primary pivotal connection, and being pivotal along a laterally inward-outward arc by way of being pivoted about the secondary pivotal connection.

The term eyeshield as used herein broadly encompasses any suitable design, whether in the form of a single piece that extends laterally in front of both eyes, or two separate pieces each of which protects one eye (and which are each connected to e.g. a brow frame as described below). In some embodiments, sidearms 200 may be pivotally connected directly to the eyeshield (e.g. to a major end thereof). However, in many embodiments it may be convenient to include a brow frame 230 (visible in various Figures) that is mounted to an upper edge of eyeshield 100 and that extends along the lateral length of the eyeshield. In such a case, each sidearm 200 may be pivotally connected to the brow frame. In the illustrated embodiment, each sidearm is pivotally connected to a first or second extending portion (236 or 237) of the brow frame, which extending portion of the brow frame extends at least generally rearwardly beyond a first major end of the eyeshield. It is emphasized that the feature of a sidearm being pivotally connected to an eyeshield by a primary pivotal connection that is “proximate” a first major end of the eyeshield, does not require that the sidearm be connected directly to the eyeshield, nor does it require that the sidearm be connected exactly to a terminal end/edge of the eyeshield (or of a brow frame). Still further, this does not require that the primary pivotal connection must necessarily be located at an extreme forward terminal end of the front part 203 of the sidearm.

If present, in some embodiments brow frame 230 may comprise a first extending portion 236 that extends at least generally rearwardly beyond a first major end of the eyeshield and to which a first sidearm is pivotally connected; and, a second extending portion 237 that extends at least generally rearwardly beyond a second, opposing major end of the eyeshield and to which a second sidearm is pivotally connected, as seen most easily in FIG. 3.

Either the brow frame, or the eyeshield itself, may be provided with a downwardly-extending nose-rest 101 at a laterally central location of the eyeshield. Nose-rest 101 may be of any suitable design, e.g. single-piece or double-piece in design. Nose-rest 101 may exhibit an uppermost point 102 as referred to earlier herein.

As shown in exemplary embodiment in FIG. 2, an earmuff-type hearing-protection apparatus 300 includes at least three major parts: a first earmuff 301, a second earmuff (not shown in any Figure, but will typically be similar or identical to first earmuff 301, and a headband. The headband connects the two earmuffs to each other and typically passes over the top of the user's head, e.g. so as to support at least a portion of the weight of apparatus 300. The headband also typically applies a biasing force to urge the two earmuffs laterally inward toward the user's head. The length of the headband, and/or the inward biasing force that it exerts, may be adjustable if desired.

Each earmuff 301 is a sound-blocking earmuff and conventionally comprises a relatively hard shell 313 (e.g., molded of any suitable plastic) that provides a sound barrier. If desired, the interior space defined by shell 313 may contain a sound-absorbing material (e.g., an acoustic foam, a sound-absorbing nonwoven, and so on) to further enhance the sound-blocking ability. Such an earmuff may be e.g. circular, elliptical, or ovoid in shape (as viewed from the vantage point of e.g. FIG. 3), having a sufficient size and dimension to cover a user's ears. If desired, such an earmuff can include electronic components e.g. so as to provide noise-cancelling capability, to receive wireless transmissions, and so on.

Each earmuff will include a compliant seal 311, which provides a seal against the side of the user's head to minimize the penetration (leakage) of sound between the earmuff and the side of the user's head. Compliant seal 311 typically relies on a soft, cushioned material that is often covered with a soft, thin layer of e.g. plastic, leather, or the like. Such a soft, thin layer can provide enhanced comfort and also may improve the sound-blocking ability (particularly if the soft, cushioned material is a relatively air-permeable material such as e.g. an open-celled foam.) The soft, cushioned nature of compliant seal 311 allows the desired seal to be achieved without applying excess pressure against the sides of the user's head.

Compliant seal 311 is positioned (e.g. mounted to, either permanently or replaceably) around the perimeter of the hard shell 313 of earmuff 301, facing inwardly (toward the side of the user's head). In use of the hearing-protection apparatus, compliant seal 311 will extend around (e.g., encircle) the entirety of the pinna 400 of the user's ear, to minimize the ability of any sound to leak between the inward surface 312 of the compliant seal and the side of the user's head 401. As noted earlier, compliant seal 311 will be sufficiently conformable that, in use, an uppermost portion of seal 311 can deform laterally outward to accommodate the presence of “earhook” 205 of the eyewear sidearm, while lower portions of the seal can deform laterally inward to maintain adequate contact with the side of the user's head, as shown in idealized representation in FIG. 5.

Eyeshield 100 may comprise any suitable layer or layers that are capable of providing at least physical protection to a user's eyes. That is, 100 eyeshield protects the user's eyes from e.g. particulate debris, splashing liquids, and so on. Eyeshield 100 may be of any suitable design. In particular, light-transmissive (e.g., transparent) portions thereof may be made of any suitable glass or polymer, e.g. polycarbonate. The transparent portions of eyeshield 100 may be non-correcting, or may comprise a vision-correcting prescription. If desired, they may include e.g. a scratch-resistant coating, and/or an anti-fog coating. If desired, they may comprise one or more UV-blocking layers or additives, may be shaded or tinted to reduce the passage of visible light, may be polarized, and so on. The curvature or lack thereof of the eyeshield may be chosen as desired. For example, an eyeshield might have a relative flat front with sideshields extending rearwardly therefrom (or, such sideshields might be attached to, or built into, e.g. a front portion of each sidearm). Or, an eyeshield may extend laterally across each eye and then sweep rearwardly in a continuous curve to provide side protection, in the exemplary manner most easily seen in FIG. 3.

Eyeshield 100, a brow frame 230 if present, and so on, may conveniently be made at least in part of injection-molded plastic pieces of any suitable thermoplastic or thermoset resin. Protective eyewear 1 may be used in any desired application, particularly those in which noise protection is needed in addition to vision protection. Such exemplary uses might include e.g. industrial operations (grinding, cutting and so on), operating of farm machinery, woodworking, and so on. In passing it is noted that the use of protective eyewear in the absence of any earmuff-type hearing-protection apparatus (e.g. with the sidearms in a lowered configuration) does not preclude the use of in-the-ear hearing protective apparatus such as earplugs and the like. If desired, protective eyewear 1 may be provided as part of a kit that includes at least one protective eyewear 1 and at least one earmuff-type hearing-protection apparatus, along with e.g. instructions for use (or a reference to a weblink where such instructions can be found). However, in the arrangements disclosed herein, sidearms 200 are not attached (permanently or removably) to hearing-protection earmuff 301 or to any part of the earmuff-type hearing-protection apparatus 300.

### LIST OF EXEMPLARY EMBODIMENTS

Embodiment 1 is a protective eyewear configured to be worn by a user, comprising: an eyeshield; a first sidearm that is pivotally connected to the eyeshield by a first primary pivotal connection that is located proximate a first major end of the eyeshield, and a second sidearm that is pivotally connected to the eyeshield by a second primary pivotal connection that is located proximate a second, opposing major end of the eyeshield, wherein the first sidearm and the second sidearm each comprise at least a first, front part and a second, rear part, and wherein at least a rearward portion of the second, rear part of each sidearm comprises a laterally-outward-and-downward-facing major surface that is a concave surface, wherein the primary pivotal connection of each sidearm is configured so that each sidearm can be pivotally moved between at least a first, lowered configuration in which the rearward portion of the rear part of the sidearm is in a maximally downward position, and a second, raised configuration in which the rearward portion of the rear part of the sidearm is in a maximally upward position, and wherein the sidearms are configured so that when the protective eyewear is worn in combination with an earmuff-type hearing-protection apparatus and when both sidearms are in the second, raised configuration, the rearward portion of the rear part of each sidearm rests atop a junction between an uppermost portion of a compliant seal of an earmuff of the hearing-protection apparatus, and a side of the user's head.

Embodiment 2 is the protective eyewear of embodiment 1 wherein the first primary pivotal connection and the second primary pivotal connection are each configured to allow their respective sidearm to pivotally move along an upward-downward arc, over a rotation angle of at least about 15 degrees. Embodiment 3 is the protective eyewear of any of embodiments 1-2 wherein the first primary pivotal connection and the second primary pivotal connection each comprise interference features that are configured so that the pivotal movement of their respective sidearm occurs in discrete increments and wherein the interference features maintain the sidearm at a rotation angle unless sufficient rotational force is applied by the user to pivotally move the sidearm to a different rotation angle. Embodiment 4 is the protective eyewear of any of embodiments 1-3 wherein the first primary pivotal connection and the second primary pivotal connection each allows its respective sidearm to be pivotally moved between at least three configurations: a first, lowered configuration; a second, upward configuration, and a third, intermediate configuration that is intermediate between the first and second configurations.

Embodiment 5 is the protective eyewear of any of embodiments 1-4 wherein at least the rearward portion of the second, rear part of each sidearm exhibits a laterally-inward facing major surface that is a generally flat surface exhibiting a major plane that is at least substantially vertically oriented when the protective eyewear is worn by a user. Embodiment 6 is the protective eyewear of any of embodiments 1-5 wherein when the protective eyewear is worn by a user in combination with an earmuff-type hearing-protection apparatus and when the first and second sidearms are each in the second, raised configuration, for each sidearm an uppermost point of the laterally-outward-and-downward-facing, concave surface of the rearward portion of the rear part of the sidearm, exhibits a positive vertical offset from a nose-rest peak of the protective eyewear, of at least about 10 mm.

Embodiment 7 is the protective eyewear of any of embodiments 1-6 wherein when the protective eyewear is worn by a user in combination with an earmuff-type hearing-protection apparatus and when the first and second sidearms are each in the second, raised configuration, for each sidearm an uppermost point of the laterally-outward-and-downward-facing, concave surface of the rearward portion of the rear part of the sidearm, exhibits a positive vertical offset from a nose-rest peak of the protective eyewear, of at least about 15 mm. Embodiment 8 is the protective eyewear of any of embodiments 1-7 wherein when the protective eyewear is worn by a user in combination with an earmuff-type hearing-protection apparatus and when the first and second sidearms are each in the second, raised configuration, for each sidearm an uppermost point of the laterally-outward-and-downward-facing, concave surface of the rearward portion of the rear part of the sidearm, exhibits a positive vertical offset from a nose-rest peak of the protective eyewear, of at least about 20 mm.

Embodiment 9 is the protective eyewear of any of embodiments 1-8 wherein when the protective eyewear is worn by a user in combination with an earmuff-type hearing-protection apparatus and when the first and second sidearms are each in the second, raised configuration, an uppermost point of the laterally-outward-and-downward-facing, concave surface of the rearward portion of the rear part of each sidearm, is positively vertically offset at least 10 mm above an uppermost point of the junction of the pinna of the user with the side of the head of the user. Embodiment 10 is the protective eyewear of any of embodiments 1-9 wherein the laterally-outward-and-downward-facing, concave surface of the rearward portion of the rear part of each sidearm exhibits an average radius of curvature of from about 10 mm to about 20 mm. Embodiment 11 is the protective eyewear of embodiment 10 wherein the rearward portion of the rear part of the sidearm exhibits a length and wherein the laterally-outward-and-downward-facing, concave surface of the rearward portion of the rear part of each sidearm exhibits a radius of curvature that does not deviate more than plus or minus 20% from an average value over the length of the rearward portion.

Embodiment 12 is the protective eyewear of any of embodiments 1-11 wherein the rearward portion of the rear part of each sidearm is in the form of an arch with a radius of curvature of from about 25 mm to about 40 mm. Embodiment 13 is the protective eyewear of any of embodiments 1-12 wherein for each sidearm, the first, front part and the second, rear part of the sidearm are slidably connected with each other and are slidably movable relative to each other so that the rear part of the sidearm can be moved rearward relative to the front part of the sidearm to increase the frontward-rearward length of the sidearm by at least about 5 mm from its shortest length. Embodiment 14 is the protective eyewear of embodiment 13 wherein for each sidearm, the slidable connection between the first, front part and the second, rear part of the sidearm comprises interference features that are configured so that the slidable motion of the rear part relative to the front part occurs in discrete increments and wherein the interference features serve to maintain the rear part of the sidearm at a location unless sufficient forward or rearward force is applied by the user to slidably move the rear part of the sidearm to a different location relative to the front part of the sidearm.

Embodiment 15 is the protective eyewear of any of embodiments 1-14 wherein each sidearm further comprises a secondary pivotal connection that allows at least the rear part of the sidearm to be rotated inwardly so that the sidearms of the protective eyewear can be placed in an inwardly-folded configuration. Embodiment 16 is the protective eyewear of embodiment 15 wherein for each sidearm, a first, forward end of the front part of the sidearm is pivotally connected to the eyeshield by a primary pivotal connection, a second, rearward end of the front part of the sidearm is slidably connected to the rear part of the sidearm; and, the secondary pivotal connection is at a location of the front part of the sidearm that is between the first, forward end of the front part of the sidearm and the second, rearward end of the front part of the side arm.

Embodiment 17 is the protective eyewear of any of embodiments 15-16 wherein the rear part of each sidearm exhibits two-axis pivotability, being pivotal along an upward-downward arc by way of being pivoted about the primary pivotal connection, and being pivotal along a laterally inward-outward arc by way of being pivoted about the secondary pivotal connection. Embodiment 18 is the protective eyewear of any of embodiments 1-17 wherein the protective eyewear comprises a brow frame that is mounted to an upper edge of the eyeshield and that extends along the length of the eyeshield, and wherein the brow frame comprises a downwardly-extending nose-rest at a laterally central location of the eyeshield, wherein the brow frame comprises a first extending portion that extends at least generally rearwardly beyond a first major end of the eyeshield and to which the first sidearm is pivotally connected; and, a second extending portion that extends at least generally rearwardly beyond a second, opposing major end of the eyeshield and to which the second sidearm is pivotally connected. Embodiment 19 is a kit comprising at least one protective eyewear of any of embodiments 1-18, packaged along with at least one earmuff-type hearing-protection apparatus.

It will be apparent to those skilled in the art that the specific exemplary elements, structures, features, details, configurations, etc., that are disclosed herein can be modified and/or combined in numerous embodiments. All such variations and combinations are contemplated by the inventor as being within the bounds of the conceived invention, not merely those representative designs that were chosen to serve as exemplary illustrations. Thus, the scope of the present invention should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially, and derivatives thereof). To the extent that there is any conflict or discrepancy between this specification as written and the disclosure in any document incorporated by reference herein, this specification as written will control.

What is claimed is:

1. A protective eyewear configured to be worn by a user, comprising:
an eyeshield;
a first sidearm that is pivotally connected to the eyeshield by a first primary pivotal connection that is located proximate a first major end of the eyeshield, and
a second sidearm that is pivotally connected to the eyeshield by a second primary pivotal connection that is located proximate a second, opposing major end of the eyeshield,
wherein the first sidearm and the second sidearm each comprise at least a first, front part and a second, rear part, and wherein at least a rearward portion of the second, rear part of each sidearm comprises a laterally-outward-and-downward-facing major surface that is a concave surface,
wherein the primary pivotal connection of each sidearm is configured so that each sidearm can be pivotally moved between at least a first, lowered configuration in which the rearward portion of the rear part of the sidearm is in a maximally downward position, and a second, raised configuration in which the rearward portion of the rear part of the sidearm is in a maximally upward position,
and wherein the sidearms are configured so that when the protective eyewear is worn in combination with an earmuff-type hearing-protection apparatus and when both sidearms are in the second, raised configuration, the rearward portion of the rear part of each sidearm rests atop a junction between an uppermost portion of a compliant seal of an earmuff of the hearing-protection apparatus, and a side of the user's head.

2. The protective eyewear of claim 1 wherein the first primary pivotal connection and the second primary pivotal connection are each configured to allow their respective sidearm to pivotally move along an upward-downward arc, over a rotation angle of at least about 15 degrees.

3. The protective eyewear of claim 1 wherein the first primary pivotal connection and the second primary pivotal connection each comprise interference features that are configured so that the pivotal movement of their respective sidearm occurs in discrete increments and wherein the interference features maintain the sidearm at a rotation angle unless sufficient rotational force is applied by the user to pivotally move the sidearm to a different rotation angle.

4. The protective eyewear of claim 1 wherein the first primary pivotal connection and the second primary pivotal connection each allows its respective sidearm to be pivotally moved between at least three configurations: a first, lowered configuration; a second, upward configuration, and a third, intermediate configuration that is intermediate between the first and second configurations.

5. The protective eyewear of claim 1 wherein at least the rearward portion of the second, rear part of each sidearm exhibits a laterally-inward facing major surface that is a generally flat surface exhibiting a major plane that is at least substantially vertically oriented when the protective eyewear is worn by a user.

6. The protective eyewear of claim 1 wherein when the protective eyewear is worn by a user in combination with an earmuff-type hearing-protection apparatus and when the first and second sidearms are each in the second, raised configuration, for each sidearm an uppermost point of the laterally-outward-and-downward-facing, concave surface of the rearward portion of the rear part of the sidearm, exhibits a positive vertical offset from a nose-rest peak of the protective eyewear, of at least about 10 mm.

7. The protective eyewear of claim 1 wherein when the protective eyewear is worn by a user in combination with an earmuff-type hearing-protection apparatus and when the first and second sidearms are each in the second, raised configuration, for each sidearm an uppermost point of the laterally-outward-and-downward-facing, concave surface of the rearward portion of the rear part of the sidearm, exhibits a positive vertical offset from a nose-rest peak of the protective eyewear, of at least about 15 mm.

8. The protective eyewear of claim 1 wherein when the protective eyewear is worn by a user in combination with an earmuff-type hearing-protection apparatus and when the first and second sidearms are each in the second, raised configuration, for each sidearm an uppermost point of the laterally-outward-and-downward-facing, concave surface of the rearward portion of the rear part of the sidearm, exhibits a positive vertical offset from a nose-rest peak of the protective eyewear, of at least about 20 mm.

9. The protective eyewear of claim 1 wherein when the protective eyewear is worn by a user in combination with an earmuff-type hearing-protection apparatus and when the first and second sidearms are each in the second, raised configuration, an uppermost point of the laterally-outward-and-downward-facing, concave surface of the rearward portion of the rear part of each sidearm, is positively vertically offset at least 10 mm above an uppermost point of the junction of the pinna of the user with the side of the head of the user.

10. The protective eyewear of claim 1 wherein the laterally-outward-and-downward-facing, concave surface of the rearward portion of the rear part of each sidearm exhibits an average radius of curvature of from about 10 mm to about 20 mm.

11. The protective eyewear of claim 10 wherein the rearward portion of the rear part of the sidearm exhibits a length and wherein the laterally-outward-and-downward-facing, concave surface of the rearward portion of the rear part of each sidearm exhibits a radius of curvature that does not deviate more than plus or minus 20% from an average value over the length of the rearward portion.

12. The protective eyewear of claim 1 wherein the rearward portion of the rear part of each sidearm is in the form of an arch with a radius of curvature of from about 25 mm to about 40 mm.

13. The protective eyewear of claim 1 wherein for each sidearm, the first, front part and the second, rear part of the sidearm are slidably connected with each other and are slidably movable relative to each other so that the rear part of the sidearm can be moved rearward relative to the front part of the sidearm to increase the frontward-rearward length of the sidearm by at least about 5 mm from its shortest length.

14. The protective eyewear of claim 13 wherein for each sidearm, the slidable connection between the first, front part and the second, rear part of the sidearm comprises interference features that are configured so that the slidable motion of the rear part relative to the front part occurs in discrete increments and wherein the interference features serve to maintain the rear part of the sidearm at a location unless sufficient forward or rearward force is applied by the user to slidably move the rear part of the sidearm to a different location relative to the front part of the sidearm.

15. The protective eyewear of claim 1 wherein each sidearm further comprises a secondary pivotal connection that allows at least the rear part of the sidearm to be rotated inwardly so that the sidearms of the protective eyewear can be placed in an inwardly-folded configuration.

16. The protective eyewear of claim 15 wherein for each sidearm, a first, forward end of the front part of the sidearm is pivotally connected to the eyeshield by a primary pivotal connection, a second, rearward end of the front part of the sidearm is slidably connected to the rear part of the sidearm; and, the secondary pivotal connection is at a location of the front part of the sidearm that is between the first, forward end of the front part of the sidearm and the second, rearward end of the front part of the side arm.

17. The protective eyewear of claim 15 wherein the rear part of each sidearm exhibits two-axis pivotability, being pivotal along an upward-downward arc by way of being pivoted about the primary pivotal connection, and being pivotal along a laterally inward-outward arc by way of being pivoted about the secondary pivotal connection.

18. The protective eyewear of claim 1 wherein the protective eyewear comprises a brow frame that is mounted to an upper edge of the eyeshield and that extends along the length of the eyeshield, and wherein the brow frame comprises a downwardly-extending nose-rest at a laterally central location of the eyeshield, wherein the brow frame comprises a first extending portion that extends at least generally rearwardly beyond a first major end of the eyeshield and to which the first sidearm is pivotally connected; and, a second extending portion that extends at least generally rearwardly beyond a second, opposing major end of the eyeshield and to which the second sidearm is pivotally connected.

19. A kit comprising at least one protective eyewear of claim 1, packaged along with at least one earmuff-type hearing-protection apparatus.
